SEQUENCE LISTING (4 sequences)

SEQ ID NO: 1 - DNA, 276 bp, Homo sapiens
  gene (1)..(276): nucleotide sequence of UQCRH
atgggactggaggacgagcaaaagatgcttaccgaatccggagatcctgaggaggaggaagaggaagaggaggaattagtggatcccctaacaacagtgagagagcaatgcgagcagttggagaaatgtgtaaaggcccgggagcggctagagctctgtgatgagcgtgtatcctctcgatcacatacagaagaggattgcacggaggagctctttgacttcttgcatgcgagggaccattgcgtggcccacaaactctttaacaacttgaaataa

SEQ ID NO: 2 - protein, 91 aa, Homo sapiens
  PEPTIDE (1)..(91): amino acid sequence of UQCRH
MGLEDEQKMLTESGDPEEEEEEEEELVDPLTTVREQCEQLEKCVKARERLELCDERVSSRSHTEEDCTEELFDFLHARDHCVAHKLFNNLK

SEQ ID NO: 3 - DNA, 20 bp, Artificial Sequence
  forward primer for UQCRH
atgggactggaggacgagca

SEQ ID NO: 4 - DNA, 20 bp, Artificial Sequence
  reverse primer for UQCRH
aagagtttgtgggccacgca

(19) United States
(12) Patent Application Publication
(10) Pub. No.: US 2012/0034235 A1
(43) Pub. Date: Feb. 9, 2012

(54) Marker for Liver-Cancer Diagnosis and Recurrence and Survival Prediction, a Kit Comprising the Same, and Prognosis Prediction in Liver-Cancer Patients Using the Marker

(75) Inventors: Kee Ho Lee, Seoul (KR); Eun Ran Park, Seoul (KR); Pu Hyeon Cha, Seoul (KR); Sang Bum Kim, Seoul (KR); Sun Hoo Park, Seoul (KR); Dong Hyoung Lee, Seoul (KR); Seon Rang Woo, Seoul (KR); Chul Ju Han, Seoul (KR); Yong Ho Ham, Seoul (KR); Eun Ju Lee, Seoul (KR); Myoung Jin Park, Seoul (KR); Sang Gu Hwang, Seoul (KR); Hyun Jin Shin, Seoul (KR)

(73) Assignee: KOREA INSTITUTE OF RADIOLOGICAL & MEDICAL SCIENCES, Seoul (KR)

(21) Appl. No.: 13/145,773

(22) PCT Filed: Jan. 22, 2010
(86) PCT No.: PCT/KR2010/000442

§ 371 Date: Oct. 4, 2011

(30) Foreign Application Priority Data
Jan. 22, 2009 (KR) 10-2009-0005693

(51) Int. Cl. A61K 39/395; C12Q 1/26; A61P 35/00; C40B 40/10; C40B 30/04; C12Q 1/68; G01N 33/573

(52) U.S. Cl. 424/158.1; 435/6.18; 435/25; 435/6.11; 435/7.4; 435/6.12; 506/18; 506/9

(57) ABSTRACT

A composition for detecting a marker for the diagnosis or prognosis of liver cancer is disclosed. The composition includes an agent capable of assessing the expression level of UQCRH (ubiquinol-cytochrome c reductase hinge protein). In addition, a kit having the composition, a microarray for the diagnosis of liver cancer using the marker, and a method for detecting the marker, and predicting recurrence following surgery in liver cancer patients are disclosed. The marker is able to contribute to the early diagnosis of liver cancer and prediction of recurrence following surgery and survival of liver cancer patients who have undergone hepatic resection, and also is significant for being a promising therapeutic target for liver cancer.

### BACKGROUND OF THE INVENTION

1. Field of the Invention

The present invention relates to a composition for detecting a diagnostic marker for liver cancer which comprises an agent capable of assessing the expression level of UQCRH (ubiquinol-cytochrome c reductase hinge protein), a kit comprising the composition, a microarray for the diagnosis of liver cancer using the marker and a method for detecting the marker, and evaluation of its efficacy as a therapeutic target for liver cancer.

2. Description of the Related Art

Liver cancer (hepatocellular carcinoma) is one of the most malignant tumors worldwide, and more than five hundred thousand people worldwide, concentrated in Asia and sub-Saharan Africa, die from liver cancer each year. A risk factor for liver cancer is known to be chronic infection of Hepatitis B or C virus. However, the molecular mechanism in hepatocellular carcinoma cells still remain unclear and thus, effective diagnostic markers for liver cancer have not been developed yet. In addition, hepatic resection is a therapeutic option for patients with non-metastatic liver cancer at an early stage. At present, about 20% of total liver cancer patients have undergone hepatic resection. However, their long-term survival rate is not high, and many patients die within 1 year after surgery. In addition, survival rate is predicted based on clinical-pathological analysis, but it is not a validated and generalized way that can be applied to all liver cancer patients. Therefore, there is an urgent need for the development of an effective diagnostic marker for liver cancer and a prognostic marker after hepatic resection.

Previous studies to understand the molecular mechanisms of liver cancer have focused on single genes, and recent studies have revealed that genes such as mutated p53, beta-catenin, AXIN 1, p21 (WAF1/CIP1) and p27 Kip are involved in hepatocarcinogenesis. However, changes in the single genes do not accurately reflect external and clinical manifestations of liver cancer patients. Thus, in addition to the previous studies on the related genes, a new approach is needed to understand molecular diversity in hepatocellular carcinoma cells of each individual. In this regard, cDNA microarray technology is a new technology capable of simultaneously identifying expression of tens of thousands of genes in a single assay over a single gene expression, and has been applied to cancer research including liver cancer. cDNA microarray technology allow the identification of genes intimately involved in carcinogenesis and proteins or enzymes expressed therefrom, thereby providing cancer diagnosis at a molecular level. Although it is difficult to make an accurate diagnosis of liver cancer based on pathological data, a difference in the molecular expression profiles enables discrimination between non-cancerous liver tissue and liver cancer tissue, because a purified set of genes involved in hepatocarcinogenesis can be chosen on the basis of the difference in the molecular expression profile. Further, an appropriate target in need can be easily identified, and thus it is expected to maximize therapeutic effects on liver cancer.

To investigate genes involved in human cancer including liver cancer by microarray, recent studies have developed an unsupervised clustering algorithm and a supervised algorithm which are very useful in the analysis of gene expression patterns. Problematically, it is difficult to provide statistical accuracy of the results and accurate profiles of expressed genes from much information of the given target by the unsupervised clustering method. Currently, there are only several reports about the importance of supervised learning algorithm employed for cancer classification and clinical data of cancer patients.

Accordingly, the present inventors have made an effort to develop a marker for the diagnosis of liver cancer and a marker for the prognosis of liver cancer after hepatic resection at a molecular level. From the results of cDNA microarray, they demonstrated that UQCRH is a gene highly expressed in hepatocellular carcinoma cells and tissues, thereby completing the present invention.

### SUMMARY OF THE INVENTION

Therefore, an object of the present invention is to provide a composition for detecting a marker for the diagnosis or prognosis of liver cancer, comprising an agent capable of assessing the expression level of UQCRH (ubiquinol-cytochrome c reductase hinge protein).

Another object of the present invention is to provide a kit for detecting a marker for the diagnosis or prognosis of liver cancer, comprising the composition.

Still another object of the present invention is to provide a microarray for the diagnosis of liver cancer, comprising UQCRH (ubiquinol-cytochrome c reductase hinge protein).

Still yet another object of the present invention is to provide a method for detecting the liver cancer marker, UQCRH (ubiquinol-cytochrome c reductase hinge protein).

Still yet another object of the present invention is to provide use of the marker of the present invention for the prediction of recurrence following surgery and survival of liver cancer patients who have undergone hepatic resection.

Still yet another object of the present invention is to evaluate efficacy of the marker of the present invention as a therapeutic target for liver cancer by analysis of UQCRH expression of liver cancer patients and their survival rate.

### BRIEF DESCRIPTION OF THE DRAWINGS

FIG. 1 illustrates the mRNA reverse transcription, hybridization on DNA chip and analysis process for expression profiles of a diagnostic gene for liver cancer;

FIG. 2 shows the gene sequence (SEQ ID NO. 1) and protein sequence (SEQ ID NO. 2) of the selected liver cancer gene, UQCRH;

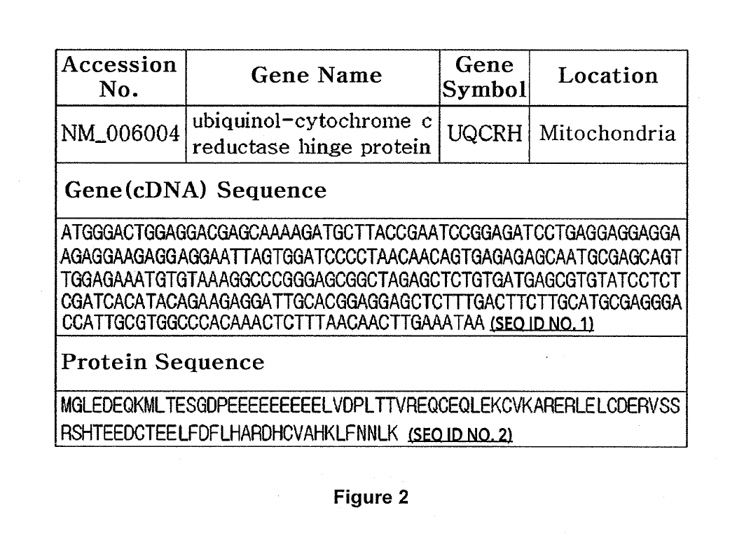

FIG. 3 shows the sequence and location of RT-PCR primers used to identify expression of the selected diagnostic gene for liver cancer UQCRH and RT-PCR conditions;

FIG. 4 is the RT-PCR results showing expression of the selected liver cancer-specific gene UQCRH in 30 tumor tissues, and comparison of the expression values between normal tissues and tumor tissues;

FIG. 5 shows the survival curves of liver cancer patients with higher and lower UQCRH expression, plotted based on the RT-PCR results of FIG. 4;

FIG. 6 shows the relative UQCRH expression of tumor tissue to normal tissue, based on Real-time PCR results;

FIG. 7 shows the survival graphs of liver cancer patients after surgery, in which the groups are divided into high expression and low expression groups, and each group has at least a 150% increased expression value than that of non-tumor tissue, based on Real-time PCR results;

FIG. 8 shows the survival graphs of liver cancer patients after surgery, in which the groups are divided into high expression and low expression groups, and each group has at least a 200% increased value, based on Real-time PCR results of FIG. 7;

FIG. 9 shows the survival graphs of liver cancer patients after surgery, in which the groups are divided into three groups according to expression values, based on the Real-time PCR results of FIG. 6; and

FIG. 10 shows the survival graphs of liver cancer patients excluding correlation with other diseases, in which the groups are divided into high expression and low expression groups, and each group has at least a 200% increased expression value than that of non-tumor tissue, based on Real-time PCR results of FIG. 6.

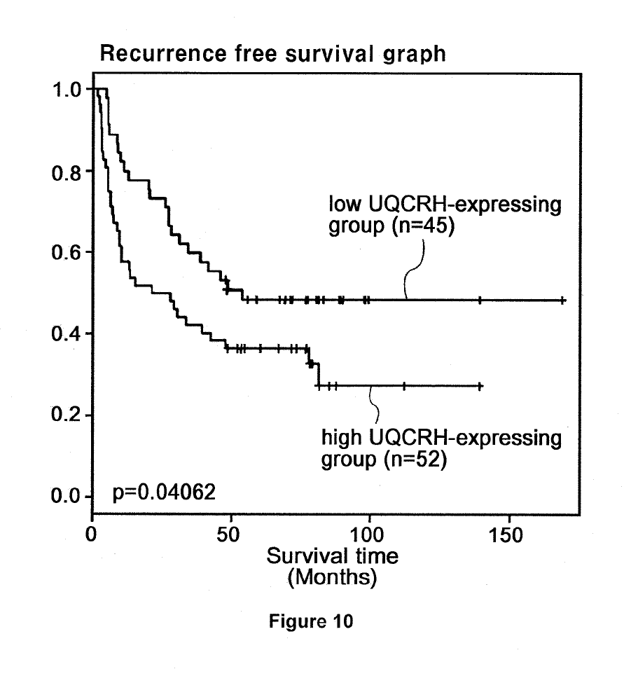

### DESCRIPTION OF THE PREFERRED EMBODIMENTS

To achieve the objects of the present invention, an aspect of the present invention relates to a composition for detecting a marker for the diagnosis or prognosis of liver cancer, comprising an agent capable of assessing the expression level of UQCRH (ubiquinol-cytochrome c reductase hinge protein).

The term “diagnosis”, as used herein, means confirmation of a pathological state or characteristic. With respect to the objects of the present invention, the diagnosis is to confirm the incidence of liver cancer.

The term “prognosis”, as used herein, refers to the prediction of the likelihood of liver cancer-attributable death or progression, including recurrence following hepatic resection, metastatic spread, and drug resistance. With respect to the objects of the present invention, the “prognosis” thus means prediction of the survival rate of liver cancer patients, and preferably prognosis of liver cancer patients who have undergone hepatic resection.

The term “prediction”, as used herein, refers to the likelihood that a patient will survive in either favorable or unfavorable response to surgical removal, chemotherapy or radiation therapy.

The term “diagnosis marker, marker for diagnosis, or diagnostic marker”, as used herein, means a material capable of distinguishing hepatocellular carcinoma cells from normal cells, and may include an organic biomolecule such as a polypeptide, a nucleic acid (e.g., mRNA etc.), a lipid, a glycolipid, a glycoprotein, and a sugar (monosaccharide, disaccharide, oligosaccharide etc.), which is expressed at a higher or lower level, as compared to its level in normal cells. With respect to the objects of the present invention, the diagnostic marker for liver cancer of the present invention is a UQCRH (ubiquinol-cytochrome c reductase hinge protein) gene, which is highly expressed in hepatocellular carcinoma cells, as compared to the normal liver cells.

The term “prognostic marker”, as used herein, means a material capable of assessing disease progression, survival, or disease-free survival after treatment of liver cancer such as hepatic resection, and may include an organic biomolecule such as a polypeptide, a nucleic acid (e.g., mRNA etc.), a lipid, a glycolipid, a glycoprotein, and a sugar (monosaccharide, disaccharide, oligosaccharide etc.). With respect to the objects of the present invention, the prognostic marker for liver cancer of the present invention is a UQCRH (ubiquinol-cytochrome c reductase hinge protein) gene.

UQCRH (ubiquinol-cytochrome c reductase hinge protein) is a gene encoding mitochondrial hinge protein, and functions to bind to cytochrome c. UQCRH is also called QCR6 and MGC111572, and information on the gene and protein are available from NCBI (National Center for Biotechnology Information) (GeneID: 7388, NM_{—}006004). Herein, the gene and amino acid sequences of UQCRH are represented by SEQ ID NOs. 1 and 2, respectively. There have been no reports of UQCRH in the studies on liver cancer, and its correlation with diagnosis and prognosis of other cancer has never been reported. Further, there are no clinical reports.

By the following demonstration, the present inventors have established that UQCRH can be used as a marker for the diagnosis of liver cancer and prediction of recurrence and survival of liver cancer patients.

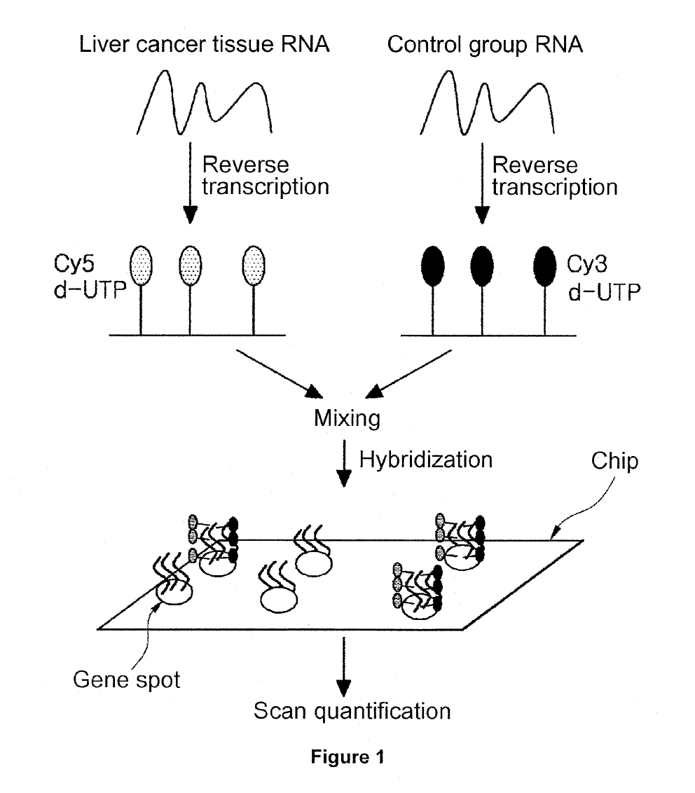

Specifically, 146 tumor tissues were collected from liver cancer patients who have undergone surgery and RNAs were isolated and purified therefrom. Normal liver tissues were collected from five non-liver cancer patients, and RNAs purified therefrom were used as a control RNA. The RNAs and control RNAs isolated from the tissues were subjected to reverse transcription to prepare cDNAs, and during this procedure, Cy5-dUTP and Cy3-dUTP were bound to cDNA, respectively (see FIG. 1). From the hybridized DNA chip, the intensities of Cy3 and Cy5 were measured at each spot using a laser scanner. The relative ratio of the two fluorescence intensities were quantified using an IMAGENE program to assess the expression level.

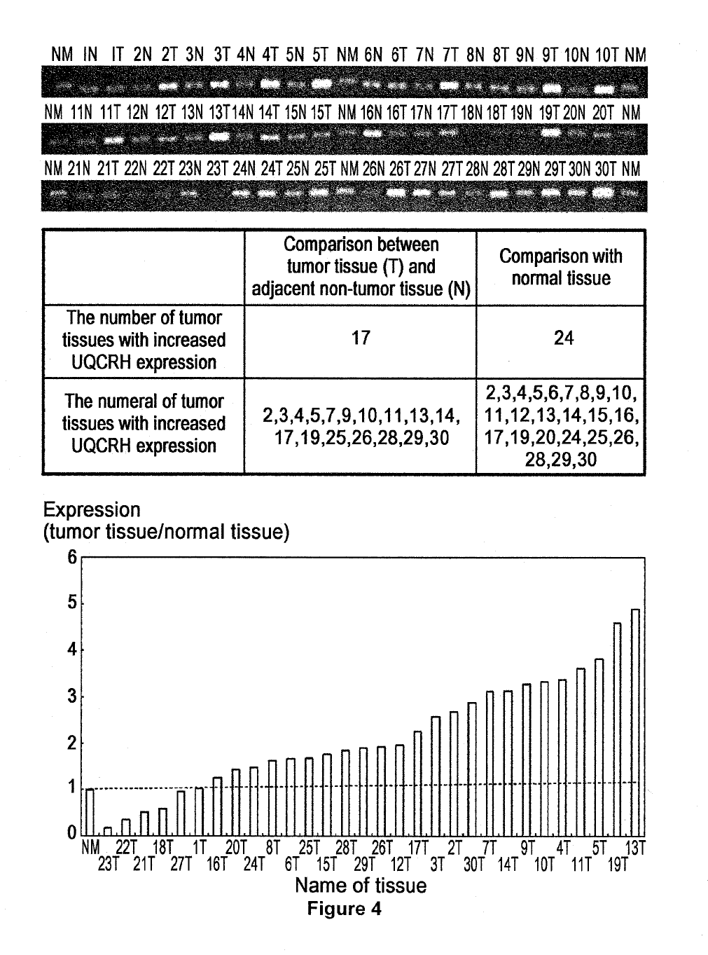

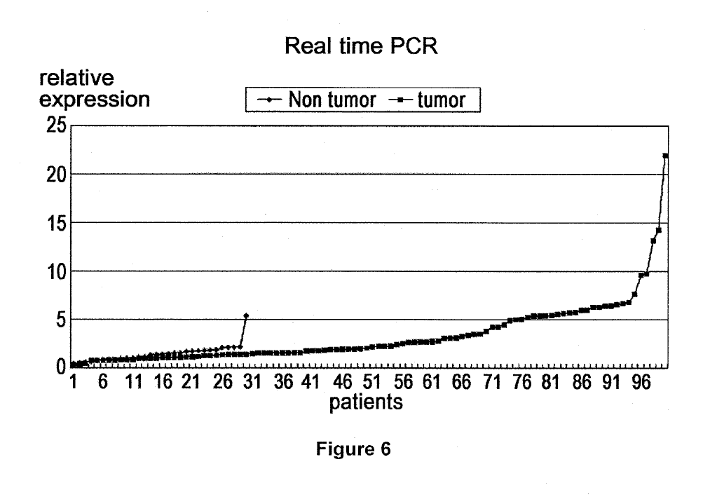

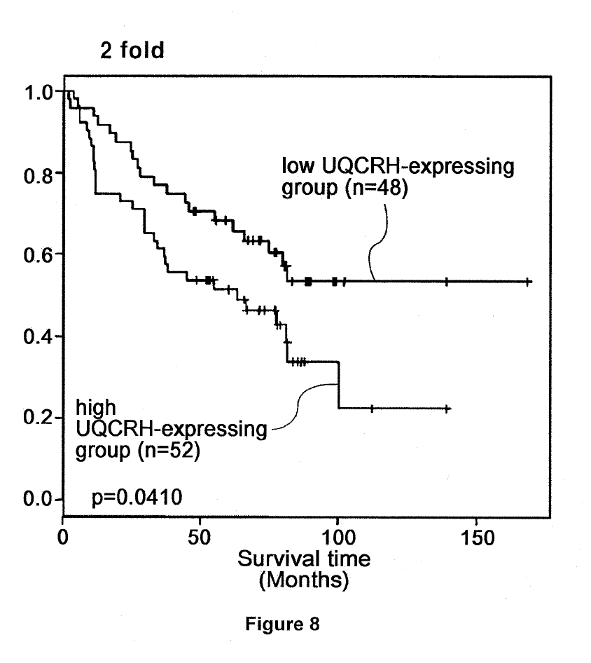

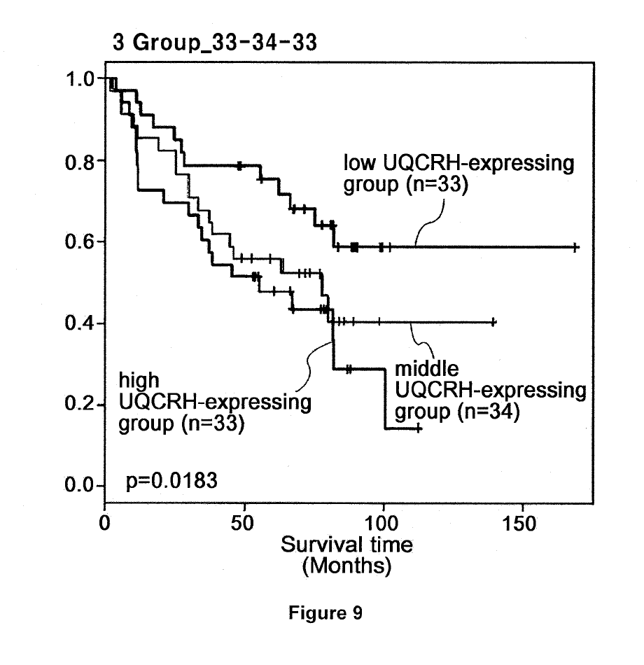

The quantified expression levels were standardized by a normalization process. Based on data of the normalized expression levels, genes with survival p value of 0.05 or less were selected. By comparison of cy3 and cy5 intensities, the genes showing higher expression level in tumor tissues (cy5) than normal tissues (cy3) were selected therefrom, and then genes with median IQR value of 0.43 or more on the microarray, in which IQR shows a difference in the expression level of a gene, were selected. In order to confirm the expression of the selected genes, RNAs were extracted from each of the 30 tumor tissues and adjacent non-tumor tissues, and 5 normal liver tissues, and cDNAs were synthesized by reverse transcription. The result of RT-PCR (reverse transcriptase polymerase chain reaction) to examine the UQCRH expression showed that its expression was remarkably increased in tumor tissues, compared to normal tissues and non-tumor tissues, and this result is identical to that of microarray (FIG. 4). Taq-Man probes for UQCRH were prepared based on cDNAs extracted from 100 samples of liver cancer patients and 30 samples of non-liver cancer patients, and the UQCRH expressions in the patient groups were assessed by Real-time PCR. On the basis of the results, survival rates according to the expression difference between liver cancer patients were analyzed. The results demonstrated that the patients with higher UQCRH expression level showed greatly reduced survival rates after surgery, compared to the patients with lower UQCRH expression levels (FIGS. 6 to 9).

The term “agent capable of assessing the expression level of UQCRH (ubiquinol-cytochrome c reductase hinge protein)”, as used herein, means a molecule used for the detection of markers by assessing the UQCRH expression level which is increased in the hepatocellular carcinoma cells, and preferably a marker-specific antibody, primer or probe.

The UQCRH expression level can be examined by assessing the mRNA expression level of UQCRH gene or the expression level of protein encoded thereby. The term “assessing the mRNA expression level”, as used herein, is a process of examining mRNA presence and expression level of the liver cancer marker gene in a biological sample for the diagnosis of liver cancer, and can be performed by measuring the amount of mRNA. The analysis method may include RT-PCR, competitive RT-PCR, real time RT-PCR, RNase protection assay (RPA), northern blotting, or DNA chip, but are not limited thereto. The term “assessing the protein expression level”, as used herein, is a process of examining the presence and expression level of the protein expressed from the liver cancer marker gene in a biological sample for the diagnosis of liver cancer, and can be performed by measuring the amount of protein using an antibody specific to the protein encoded by the gene. The analysis method may include Western blotting, ELISA (enzyme linked immunosorbent assay), RIA (radioimmunoassay), radioimmunodiffusion, ouchterlony immunodiffusion, rocket immunoelectrophoresis, immunohistostaining, immunoprecipitation assay, complement fixation assay, FACS and protein chip, but are not limited thereto.

The agent capable of assessing the mRNA expression level is preferably a pair of primers or a probe. The nucleic acid sequence of UQCRH gene is available from NM_{—}006004 (NCBI). Thus, on the basis of the sequence, those skilled in the art are able to design a primer or probe that specifically amplifies a specific region of the gene.

The term “primer”, as used herein, means a short nucleic acid strand having a free 3′ hydroxyl group, which is able to form a base pair with a complementary template, and functions as a starting point for amplifying the template. The primer can initiate DNA synthesis in the presence of a regent for polymerization in a suitable buffer solution, at a suitable temperature (DNA polymerase, or reverse transcriptase) and four different deoxynucleoside triphosphates. In the present invention, PCR is performed using sense and antisense primers of UQCRH polynucleotide to identify the production of a desired product, thereby diagnosing liver cancer. PCR conditions and length of sense and antisense primers can be modified on the basis of the methods known in the art.

The term “probe”, as used herein, means a fragment of nucleic acid such as an RNA or DNA, which is several to hundreds of base pairs capable of specifically binding to mRNA, and is labeled to identify the presence of specific mRNA. The probe can be prepared in a form of oligonucleotide probe, single stranded DNA probe, double stranded DNA probe, RNA probe or the like. In the present invention, hybridization is performed using a probe complementary to the UQCRH polynucleotide, and then liver cancer can be diagnosed by the hybridization result. Selection of suitable probe and hybridization conditions can be modified on the basis of the methods known in the art.

The primer or probe of the present invention can be chemically synthesized using a phosphoramidite solid support method or other conventional method. The sequence of the nucleic acid can be also modified using various methods known in the art. Non-limiting examples of such modifications include methylation, capping, substitution with one or more homologue of natural nucleotide, and modification between nucleotides, for example, modification to an uncharged linker (e.g., methyl phosphonate, phosphotriester, phosphoroamidate, and carbamate) or charged linker (e.g., phosphorothioate, phosphorodithioate).

The agent capable of assessing the protein expression level is preferably an antibody.

The term “antibody”, as used herein, is a term known in the art, and refers to a specific protein molecule that indicates an antigenic region. With respect to the objects of the present invention, the antibody refers to an antibody that specifically binds to the marker of the present invention, UQCRH. To prepare the antibody, each gene is cloned into an expression vector according to the typical method, so as to obtain a protein encoded by the marker gene, and then the antibody may be prepared from the protein according to the typical method, in which a partial peptide prepared from the protein is included, and the partial peptide of the present invention includes at least 7 amino acids, preferably 9 amino acids, and more preferably 12 amino acids or more. There is no limitation in the form of the antibody of the present invention, and a polyclonal antibody, a monoclonal antibody, or a part thereof having antigen-binding property is also included, and all immunoglobulin antibodies are included. Furthermore, the antibody of the present invention also includes special antibodies, such as a humanized antibody.

The antibodies used in the detection of liver cancer marker of the present invention include complete forms having two full-length light chains and two full-length heavy chains, as well as functional fragments of antibody molecules. The functional fragments of antibody molecules refer to fragments retaining at least an antigen-binding function, and include Fab, F(ab′), F(ab′)₂, Fv or the like.

In another aspect, the present invention relates to a kit for detecting a marker for the diagnosis of liver cancer, comprising the composition for detecting a diagnostic marker for liver cancer.

The kit of the present invention can detect the marker by determining the mRNA or protein level of the diagnostic marker for liver cancer, UQCRH. The detection kit of the present invention may comprise a primer or probe to measure the expression level of the diagnostic marker for liver cancer, an antibody selectively recognizing the marker, as well as one or more kinds of a composition, a solution, or an apparatus, which are suitable for the analysis method.

In a specific embodiment, the kit to assess the mRNA expression level of UQCRH may be a kit that includes essential elements required for performing RT-PCR. An RT-PCR kit may include test tubes or other suitable containers, reaction buffers (varying in pH and magnesium concentrations), deoxynucleotides (dNTPs), enzymes such as Taq-polymerase and reverse transcriptase, DNase, RNase inhibitor, DEPC water, and sterile water, in addition to a pair of primers specific to the marker gene, which are designed by those skilled in the art. In addition, 18s rRNA was used as a control group, and a pair of primers specific thereto may be included. The kit of the present invention may also be a kit for detecting the diagnostic marker, comprising essential elements required for performing a DNA chip. The DNA chip may include a base plate, onto which cDNAs corresponding to genes or fragments thereof are attached as a probe, and the base plate may also include cDNA corresponding to a control gene or fragments thereof.

In another specific embodiment, the kit for measuring the protein expression level of UQCRH may include a matrix, a suitable buffer solution, a coloring enzyme, or a secondary antibody labeled with a fluorescent substance, a coloring substrate or the like for the immunological detection of antibody. As for the matrix, a nitrocellulose membrane, a 96 well plate made of polyvinyl resin, a 96 well plate made of polystyrene resin, and a slide glass may be used. As for the coloring enzyme, peroxidase and alkaline phosphatase may be used. As for the fluorescent substance, FITC and RITC may be used, and as for the coloring substrate solution, ABTS (2,2′-azino-bis-(3-ethylbenzothiazoline-6-sulfonic acid)), OPD (o-phenylenediamine), or TMB (tetramethyl benzidine) may be used.

In still another aspect, the present invention relates to a microarray for the diagnosis of liver cancer, comprising the marker according to the present invention. The microarray of the present invention may be readily fabricated using the marker of the present invention by the method typically used in the art.

In still another aspect, the present invention relates to a method for detecting the liver cancer marker UQCRH by comparing the UQCRH expression level in the sample of a patient with that in a normal cell in order to provide information needed for liver cancer diagnosis.

More specifically, the gene expression can be detected at an mRNA or protein level, and the isolation of mRNA or protein from a biological sample may be achieved using a known process.

The term “biological sample”, as used herein, includes samples displaying a difference in expression level of the liver cancer marker gene UQCRH, such as tissues, cells, whole blood, serum, plasma, saliva, sputum, cerebrospinal fluid or urine, but is not limited thereto.

With the detection methods, the occurrence of liver cancer can be diagnosed by comparing the gene expression level in a patient suspected of having liver cancer to that in a normal control group. That is, the expression level of the marker of the present invention in a suspected liver cancer cell is assessed and then compared to that in a normal cell. If a significant increase in the expression level of the marker is observed in the suspected liver cancer cell, the suspected liver cancer can be finally diagnosed as liver cancer.

Analysis methods for assessing the mRNA level include, but are not limited to, reverse transcriptase polymerase chain reaction, competitive reverse transcriptase polymerase chain reaction, real-time reverse transcriptase polymerase chain reaction, RNase protection assay, Northern blotting and DNA chip assay. With the detection methods, the mRNA expression level in a patient suspected of having liver cancer is compared with that in a normal control, and the patient's suspected liver cancer is diagnosed by determining whether mRNA expression levels of the liver cancer marker gene have significantly increased.

The mRNA expression levels are preferably assessed by reverse transcriptase polymerase chain reaction using primers being specific to the gene used as a liver cancer marker, or DNA chip assay.

After the reverse transcriptase polymerase chain reaction, the products are electrophoresed, and patterns and thicknesses of bands are analyzed to determine the expression and level of mRNA from a gene used as a diagnostic marker of liver cancer while comparing its mRNA expression and level with those of a control, thereby easily diagnosing the incidence of liver cancer.

Meanwhile, the DNA chip is a DNA chip in which the liver cancer marker genes or fragments thereof are anchored at high density to a glass-like base plate. A cDNA probe labeled with a fluorescent substance at its end or internal region is prepared using mRNA isolated from a sample, and is hybridized with the DNA chip, thereby diagnosing the incidence of liver cancer.

Analysis methods for assessing the protein level include, but are not limited to, Western blotting, ELISA, radioimmunoassay, radialimmunodiffusion, Ouchterlony immunodiffusion, rocket immunoelectrophoresis, immunohistostaining, immunoprecipitation assay, complement fixation assay, FACS, and protein chip assay. With the analysis methods, a patient suspected of having liver cancer is compared with a normal control for the amount of formed antigen-antibody complexes, and the patient's suspected liver cancer can be diagnosed by evaluating whether there is a significant increase in the protein expression level of the liver cancer marker gene.

The term “antigen-antibody complexes”, as used herein, refers to binding products of a liver cancer marker protein to an antibody specific thereto. The amount of formed antigen-antibody complexes may be quantitatively determined by measuring the signal intensity of a detection label.

Preferably, the protein expression level is assessed by ELISA. Examples of ELISA include direct ELISA using a labeled antibody recognizing an antigen immobilized on a solid support, indirect ELISA using a labeled antibody recognizing a capture antibody forming complexes with an antigen immobilized on a solid support, direct sandwich ELISA using another labeled antibody recognizing an antigen in an antigen-antibody complex immobilized on a solid support, and indirect sandwich ELISA, in which another labeled antibody recognizing an antigen in an antigen-antibody complex immobilized on a solid support is reacted, and then a secondary labeled antibody recognizing another labeled antibody is used. More preferably, the protein expression levels are detected by sandwich ELISA, where a sample reacts with an antibody immobilized on a solid support, and the resulting antigen-antibody complexes are detected by adding a labeled antibody recognizing the antigen of the antigen-antibody complexes, followed by enzymatic color development, or by adding a labeled secondary antibody specific to the antibody which recognizes the antigen of the antigen-antibody complex, followed by enzymatic development. The incidence of liver cancer may be diagnosed by measuring the degree of complex formation of a liver cancer marker protein and an antibody specific thereto.

Further, the protein expression level is preferably assessed by Western blotting using one or more antibodies against the liver cancer markers. Total proteins are isolated from a sample, electrophoresed to be separated according to size, transferred onto a nitrocellulose membrane, and reacted with an antibody. The amount of proteins produced by gene expression is determined by measuring the amount of produced antigen-antibody complexes using a labeled antibody, thereby diagnosing the incidence of liver cancer. The detection methods are composed of methods of assessing expression levels of marker genes in the control and liver cancer cells. The mRNA or protein level may be expressed as an absolute (e.g., μg/ml) or relative (e.g., relative intensity of signals) difference in the amount of marker protein.

In addition, the protein expression level is preferably assessed by immunohistostaining using one or more antibodies against the liver cancer markers. Normal colon epithelial tissues and suspected liver cancer tissues were collected and fixed, and then paraffin-embedded blocks were prepared according to a widely known method. The blocks were cut into small sections (several um in thickness), and attached to glass slides to be reacted with one or more selected from the antibodies according to a known method. Subsequently, the unreacted antibodies were washed, and the reacted antibodies were labeled with one of the above mentioned detection labels, and then observed under a microscope.

It is also preferable to analyze the protein level using a protein chip, in which one or more antibodies against the liver cancer marker are arranged and fixed at a high density at predetermined positions on a substrate. In this regard, proteins are separated from a sample and hybridized with a protein chip to form an antigen-antibody complex, which is then read to examine the presence or expression level of the protein of interest, thereby diagnosing the occurrence of liver cancer.

In still another aspect, the present invention relates to a method for detecting the liver cancer marker UQCRH by comparing the UQCRH expression level in the sample of a patient who has undergone a hepatic resection with that in a normal cell in order to provide information needed for the prognosis after hepatic resection.

By the detection method, the expression pattern of prognostic gene is analyzed to predict recurrence and survival of liver cancer patients who have undergone hepatic resection, and on the basis of the prediction, it can be used as a target to predict the prognosis of patients.

In still another aspect, the present invention relates to a method for improving the survival of liver cancer patients by reducing the abnormal UQCRH expression level of liver cancer patient to a normal level.

More particularly, the abnormal UQCRH expression level of a liver cancer patient can be reduced to a normal level by using the UQCRH-specific antibody or functional fragment thereof or by using an oligonucleotide inhibiting the UQCRH gene expression. Preferably, the oligonucleotide may be an siRNA or shRNA against mRNA of UQCRH, or an antisense nucleic acid sequence complementary to mRNA of UQCRH.

Hereinbelow, the present invention will be described in more detail with reference to Examples. However, these Examples are for illustrative purposes only, and the invention is not intended to be limited by these Examples.

### Example 1

### Selection of Experimental Groups and Test Tissue Samples

Test samples were obtained from 98 liver cancer patients who have undergone surgery at the Korea cancer center hospital and 58 liver cancer patients who have undergone surgery in Seoul National University Hospital. All patients signed a consent form for the use of their surgical specimens and clinical and pathological data for the purpose of research. After surgical resection, all tissues were immediately frozen in liquid nitrogen, and then stored at −80° C. RNAs were isolated from the frozen tissues according to the experimental manual (RNeasy MiniElute Cleanup Kit, Qiagen). Quality of the total RNAs were determined by 28S and 18S rRNA band ratios after agarose gel electrophoresis.

### Example 2

### Analysis of cDNA Microarray Data and Gene Selection

The microarray used in the experiment was composed of total 24,094 human cDNA clones. The experimental method for microarray was performed using a 3DNA array detection kit (Genisphere Inc. PA, USA) according to the manufacturer's instructions. Thereafter, scanning was performed using a Scanarray scanner (PerkinElmer, Boston, Mass., USA). The quantified data was obtained from scanned image files using IMAGENE 4.0 (Biodiscovery, Marina del Rey, Calif., USA), and normalization was performed considering the fluorescence intensity and spot location (spatially dependent method). From the normalized microarray data, genes with survival p value of 0.05 or less were selected. By the comparison of cy3 and cy5 intensities, the genes showing higher expression level in tumor tissues (cy5) than normal tissues (cy3) were selected therefrom, and then the genes with a median IQR value of 0.43 or more on the microarray, in which IQR shows a difference in the expression level of a gene, were selected. In order to identify the expression of the selected genes, RNAs were extracted from each 30 of the tumor tissues and adjacent non-tumor tissues, and 5 normal liver tissues, and cDNAs were synthesized by reverse transcription. The result of RT-PCR (reverse transcriptase polymerase chain reaction) to examine the UQCRH expression showed that its expression was remarkably increased in tumor tissues, compared to non-tumor tissues, and this result is identical to that of the microarray. In the microarray data, higher intensity was also observed in tumor tissues (cy5) compared to the normal tissues (cy3).

### Example 3

### Identification of Prognostic Gene Expression by RT-PCR and Prediction of Liver Cancer

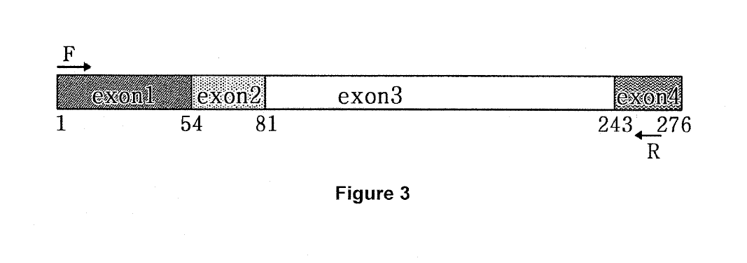

In order to indentify the UQCRH expressions of the genes selected in Example 2, RNAs were extracted from each 30 of the tumor tissues and adjacent non-tumor tissues, and 5 normal liver tissues, and cDNAs were synthesized by reverse transcription. RT-PCR (reverse transcriptase polymerase chain reaction) was performed under the condition of Table 1 to identify the UQCRH gene expression. FIG. 3 shows the location of RT-PCR primer sequences used for identification of the UQCRH gene expression.

TABLE 1

| | | | Number of |
|---|---|---|---|
| | Primer sequence | Annealing Tm | cycle in |
| Name | (5′ -> 3′) | in RT-PCR | RT-PCR |
| F | ATGGGACTGGAGGACGAGCA | 56° C. | 22 |
| | (SEQ ID NO. 3) | | |
| R | AAGAGTTTGTGGGCCACGCA | | |
| | (SEQ ID NO. 4) | | |

The results showed that increased UQCRH expression was observed in 17 tumor tissues, compared to non-tumor tissues, and its expression was remarkably increased in 24 tumor tissues, compared to normal liver tissues (FIG. 4).

### Example 4

### Prediction of Survival of Liver Cancer Patients by RT-PCR of Prognostic Gene

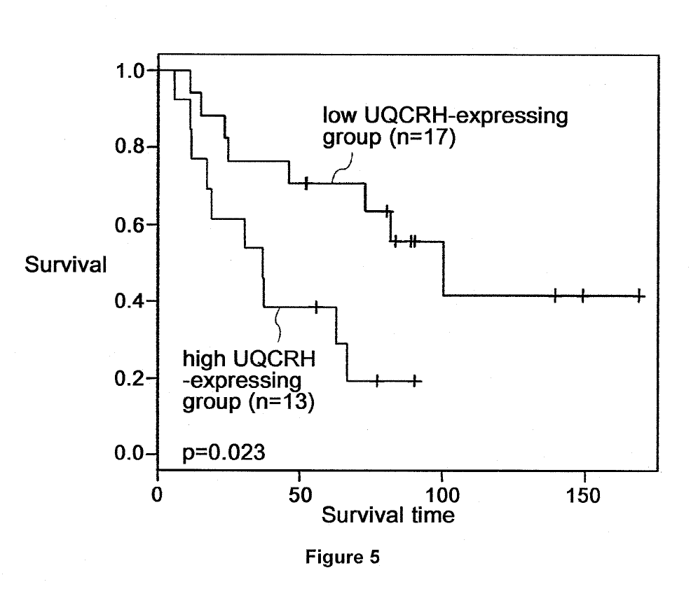

The RT-PCR results of UQCRH in 30 tumor tissues, non-tumor tissues and normal tissues were analyzed by agarose gel electrophoresis. The band intensity showing the UQCRH expression was quantified using the IMAGE J program (NIH) to obtain the quantified intensity of tumor tissues relative to that of normal tissues (Nm: normal liver mix). Groups were divided into a group having the relative value of less than 2 and a group having the relative value of 2 or more, and Kaplan-Meier curves were plotted using the R project for statistical computing, and significance was tested with a Log Rank test. The survival p-value of UQCRH showed a significant value of p=0.023, and consequently, UQCRH can be used as a prognostic marker to predict survival of liver cancer patients (FIG. 5).

### Example 5

### Prediction of Survival of Liver Cancer Patients by Real Time-PCR of Prognostic Gene

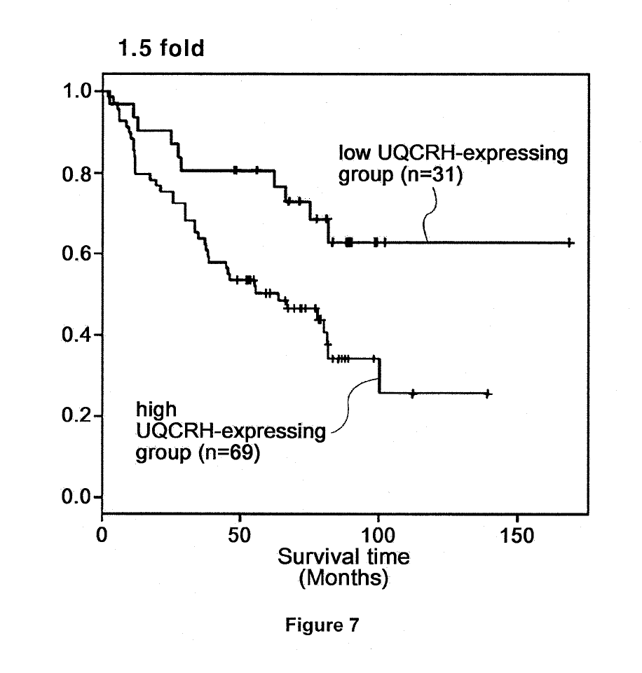

Taq-Man probes for UQCRH were prepared based on cDNAs extracted from 100 samples of liver cancer patients and 30 samples of non-liver cancer patients, and the UQCRH expressions in the patients were accurately assessed by Real-time PCR which provides more accurate data than other known methods (FIG. 6). On the basis of the results, survival rates according to the expression difference between liver cancer patients were analyzed to obtain a significant result of p=0.041. The results demonstrated that the patients with higher UQCRH expression level showed greatly reduced survival rates after surgery, compared to the patients with lower UQCRH expression levels (FIGS. 7 to 9), suggesting that UQCRH can be used as a promising therapeutic target for liver cancer.

### Effect of the Invention

The present inventors investigated genes showing higher expression in liver cancer tissues than in normal liver tissues, and selected and evaluated the gene as a diagnostic marker for liver cancer. UQCRH, the diagnostic marker for liver cancer according to the present invention, is therefore used to detect the occurrence of liver cancer with ease, and furthermore it can be utilized in the studies on hepatocarcinogenesis. In addition, it is able to contribute to the early diagnosis of liver cancer and prediction of recurrence and survival of liver cancer patients, and also is significant for being a promising therapeutic target for liver cancer.

1. A composition for detecting a marker for the diagnosis or prognosis of liver cancer, comprising an agent capable of assessing mRNA or protein expression level of UQCRH (ubiquinol-cytochrome c reductase hinge protein).

2. The composition according to claim 1, wherein the agent capable of assessing mRNA expression level of the gene is a pair of primers specifically binding to the UQCRH gene.

3. The composition according to claim 1, wherein the agent capable of assessing mRNA expression level of the gene is a probe specifically binding to the UQCRH gene.

4. The composition according to claim 1, wherein the agent capable of assessing protein expression level is an antibody specific to the UQCRH protein.

5. A kit for the diagnosis or prognosis of liver cancer, comprising the composition of claim 1.

6. The kit according to claim 5, wherein the kit is an RT-PCR kit, a DNA chip kit or a protein chip kit.

7. A microarray for the diagnosis of liver cancer, comprising UQCRH.

8. A method for detecting the liver cancer marker, UQCRH comprising:
assessing mRNA expression level of UQCRH gene or expression level of protein encoded thereby in a biological sample from a patient; and
comparing the mRNA or protein expression level with that of a normal control sample in order to provide information needed for liver cancer diagnosis.

9. The method according to claim 8, wherein the method for assessing mRNA expression level is to use a pair of primers or a probe specifically binding to the UQCRH gene.

10. The method according to claim 8, wherein the method for assessing mRNA expression level is to use any one selected from reverse transcriptase polymerase chain reaction, competitive reverse transcriptase polymerase chain reaction, real-time reverse transcriptase polymerase chain reaction, RNase protection assay, Northern blotting, and DNA chip.

11. The method according to claim 8, wherein the method for assessing protein expression level is to use an antibody specific to the corresponding protein.

12. The method according to claim 8, wherein the method for assessing protein expression level is to use any one selected from Western blotting, ELISA, radioimmunoassay, radioimmunodiffusion, ouchterlony immunodiffusion, rocket immunoelectrophoresis, immunohistostaining, immunoprecipitation assay, complement fixation assay, FACS and protein chip.

13. A method for detecting the liver cancer marker UQCRH by comparing the UQCRH expression level in a sample of a patient who has undergone a hepatic resection with that in a normal cell in order to provide information needed for the prediction of recurrence following hepatic resection and prognosis of liver cancer.

14. A method for improving the survival of liver cancer patients by reducing the abnormal UQCRH expression level of the liver cancer patient to a normal level.
